(19) United States
(12) Patent Application Publication
(10) Pub. No.: US 2005/0187239 A1
(43) Pub. Date: Aug. 25, 2005

(54) Synergistic l-methadone compositions and methods of use thereof

(76) Inventor: Gavril W Pastenark, New York, NY (US)

Correspondence Address: EDWARDS & ANGELL, LLP, P.O. BOX 55874, BOSTON, MA, 02205, US

(21) Appl. No.: 10/509,050

(22) PCT Filed: Mar. 27, 2003
(86) PCT No.: PCT/US03/09766

Related U.S. Application Data
(60) Provisional application No. 60/367,790, filed on Mar. 27, 2002.
(60) Provisional application No. 60/416,414, filed on Oct. 7, 2002.

(51) Int. Cl. A61K 31/7052; A61K 31/485

(52) U.S. Cl. 514/282; 514/23

(57) ABSTRACT

The present invention relates to pharmaceutical compositions and methods wherein L-methadone and at least one other opioid analgesic are administered in amounts effective to potentiate a synergistic antinociceptive response to moderate and severe pain, preferably with decreased dependence and tolerance potential. Synergistic potentiation of analgesia through administration of pharmaceutical compositions comprising L-methadone and at least one other opioid analgesic provides a new and improved approach to pain management.

### CROSS-REFERENCE TO RELATED APPLICATIONS

This application claims priority to U.S. application Ser. No. 60/367,790, filed on Mar. 27, 2002, and to U.S. application Ser. No. 60/416,414, filed on Oct. 7, 2002. This application makes reference to U.S. application Ser. No. 09/844,111, filed on Apr. 27, 2001, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### STATEMENT OF RIGHTS TO INVENTIONS MADE UNDER FEDERALLY SPONSORED RESEARCH

This work was supported by the government, in part, by grants from the National Institute of Drug Abuse, No. DA07242. The government may have certain rights to this invention.

### TECHNICAL FIELD

The present invention relates to pharmaceutical compositions and methods wherein L-methadone and at least one other opioid analgesic are administered in amounts effective to potentiate a synergistic antinociceptive response to moderate and severe pain, preferably with decreased dependence and tolerance potential. Synergistic potentiation of analgesia through administration of pharmaceutical compositions comprising L-methadone and at least one other opioid analgesic provides a new and improved approach to pain management. Surprisingly, synergy between L-methadone and opioid analgesics of the present invention provides new evidence that L-methadone can exert its effects through mechanisms distinct from other μ-opioid analgesics.

### BACKGROUND ART

Opiates are drugs derived from opium and include morphine, codeine and a wide variety of semi-synthetic opioid congeners derived from them and from thebaine, another component of opium. Opioids include the opiates and all agonists and antagonists with morphine-like activity and naturally occurring endogenous and synthetic opioid peptides. Morphine and other morphine-like opioid agonists are the therapeutic choice in the management of severe pain. Morphine and other opioids demonstrate potent stereoselective effects that are reversed by selective antagonists, suggesting interactions with specific membrane receptors.

Multiple classes of opioid receptors mediate the complex interactions of morphine and drugs with mixed agonist/antagonist properties. Opioid receptors comprise a family of cell surface proteins, which control a range of biological responses, including pain perception, modulation of affective behavior and motor control, autonomic nervous system regulation and neuroendocrinological function. There are three major classes of opioid receptors in the CNS, designated μ (mu), κ (kappa) and δ (delta), which differ in their affinity for various opioid ligands and in their cellular distribution. The different classes of opioid receptors are believed to serve different physiologic functions (Olson et al. (1989) Peptides 10: 1253; Lutz, R. A. and Pfister, H. P. (1992) J. Receptor Res. 12: 267-86; and Simon, E. J. (1991) Med. Res. Rev. 11: 357-74). Morphine produces analgesia primarily through the μ-opioid receptor. However, among the opioid receptors, there is substantial overlap of function as well as of cellular distribution.

The μ-opioid receptor mediates the actions of morphine and morphine-like opioids, including most clinical analgesics. In addition to morphine, several highly selective agonists exist for μ-opioid receptors, including [D-Ala²,MePhe⁴,Gly(ol)⁵]enkephalin (DAMGO), levorphanol and methadone. Differential sensitivity to antagonists, such as naloxonazine and naloxone, indicates the pharmacological distinctions between the μ-opioid receptor subtypes, μ₁ and μ₂. Several of the opioid peptides will also interact with μ-opioid receptors.

The amino acid sequences of the opioid receptors are approximately 65% identical, and they have little sequence similarity to other G-protein-coupled receptors except for somatostatin (Reisine, T. and Bell, G. I. (1993) Trends Neurosci. 16: 506-510). The regions of highest similarity in sequence are predicted to lie within the seven transmembrane-spanning regions and within the intracellular loops. Regions of amino acid sequence divergence are the amino and carboxy termini and the second and third extracellular loops.

Each receptor subtype has a characteristic pattern of expression. μ-opioid receptor MRNA is present in regions of the brain involved in pain perception and associated with morphine analgesia (Delfs et al. (1994) J Comp. Neurol. 345: 46-68); in nuclei involved in control of respiration, which is consistent with the ability of morphine to depress respiration; and in neurons where morphine has been shown to cause nausea and induce vomiting. Other consequences of μ-opioid receptor activation include miosis, reduced gastrointestinal motility, and feelings of well being or euphoria (Pasternak, G. W. (1993) Clin. Neuropharmacol. 16: 1-18). The pattern of μ-opioid receptor mRNA expression correlates with the brain centers involved in mediating the biological actions of morphine and μ-selective agonists. δ-opioid receptor mRNA is found in the dorsal horn of the spinal cord. κ₁-opioid receptor niRNA is expressed in the hypothalamic regions, which may account for many of the neuroendocrine effects of the κ-selective agonists.

In recent years, a number of μ-opioid receptor subtypes have been proposed. The first suggestion of μ₁ and μ₂ receptor subtypes came from a combination of binding and pharmacological studies based on the antagonists, naloxonazine and naloxazone (Wolozin, B. L. and Pasternak, G. W. (1981) Proc. Natl. Acad. Sci. U.S.A. 78: 6181-6185; Reisine and Pasternak (1996) Goodman & Gilman's The pharmacological basis of therapeutics, Ninth Edition (Hardman et al. eds.) McGraw-Hill pp. 521-555; and Pasternak (1993). To date, only a single μ-receptor gene, MOR-1, has been identified (Min, B. H. et al. (1994) Proc. Natl. Acad. Sci. U.S.A. 91: 9081-9085; Giros, B. et al. (1995) Life Sci. 56: PL369-375; and Liang, Y. et al. (1995) Brain Res. 679: 82-8). The MOR-1 cDNA consists of exons 1-4, which total 1610 bp in length and encode 398 amino acids. More recently, pharmacological and molecular differences between morphine and its metabolite morphine-6β-glucuronide (M6G) have suggested yet another μ-opioid receptor subtype (Pasternak, G. W. and Standifer, K. M. (1995) Trends Pharm. Sci. 16: 344-350; Rossi, G. C. et al. (1995) FEBS Lett. 369(2-3): 192-6; and Rossi, G. C. et al. (1996) Neurosci. Lett. 216: 1-4).

The first two MOR-1 splice variants identified were the human MOR-1A and the rat MOR-1B_{S} (Bare, L. A. et al. (1994) FEBS Lett. 354(2): 213-6; and Zimprich, A. et al. (1995) FEBS Lett 359(2-3): 142-6). The hMOR-1A splice variant consists of exons 1, 2, 3 and a new exon 3a, and was determined to possess ligand binding characteristics similar to the full-length MOR-1 (Bare et al. 1994). The rMOR-1B_{S} splice variant consists of exons 1, 2, 3 and a new exon 5, and like hMOR-1A, differs from MOR-1 only in length and amino acid composition at the carboxy-terminal tail (Zimprich et al. 1995). MOR-1B_{S} has affinity to opioid compounds similar to that of MOR-1, but is much more resistant to agonist-induced desensitization than MOR-1. The C-terminal differences between MOR-1 and MOR-1A or MOR-1B_{S} could have effects on receptor coupling or receptor transport and localization. At least 15 μ-opioid receptor splice variants have now been identified and characterized, and at least 10 display high affinity and selectivity for 1 opioids in receptor binding assays (Pan et al., 1999, 2000, 2001). An additional four show splicing leading to differences in the C-terminus, similar to the differences seen with MOR-1A and MOR-1B. The MOR-1 splice variants are potential targets for the modulation of physiological effects resulting from μ-opioid receptor activity.

There are many compounds with pharmacological properties similar to those produced by morphine, but none has proven to be clinically superior in relieving pain. The effects of morphine on humans are relatively diverse and include analgesia, drowsiness, changes in mood, respiratory depression, decreased gastrointestinal motility, nausea, vomiting, and alterations of the endocrine and autonomic nervous systems. (Pasternak, G. W., (1993). Clin. Neuropharmacol. 16:1). Doses of morphine need to be tailored based on individual sensitivity to the drug and the pain-sparing needs of the individual. For instance, the typical initial dose of morphine (10 mg/70 kg) relieves post-operative pain satisfactorily in only two-thirds of patients. Likewise, responses of an individual patient may vary dramatically with different morphine-like drugs and patients may have side effects with one such drug and not another. For example, some patients who are unable to tolerate morphine may have no problems with an equi-analgesic dose of methadone, another μ-opioid agonist. The mechanisms underlying variations in individual responses to morphine and morphine-like agonists have not been defined.

Side effects resulting from the use of morphine range from mild to life threatening. Morphine causes constriction of the pupil by an excitatory action on the parasympathetic nerve innervating the pupil. Morphine depresses the cough reflex through inhibitory effects on the cough centers in the medulla. Nausea and vomiting occur in some individuals through direct stimulation of the chemoreceptor trigger zone for emesis. Therapeutic doses of morphine also result in peripheral vasodilatation, reduced peripheral resistance and an inhibition of baroreceptor reflexes in the cardiovascular system. Additionally, morphine provokes the release of histamines, which can cause hypotension. Morphine depresses respiration, at least in part by direct effects on the brainstem regulatory systems. In humans, death from morphine poisoning is nearly always due to respiratory arrest. Opioid antagonists can produce a dramatic reversal of severe respiratory depression and naloxone is currently the treatment of choice. High doses of morphine and related opioids can produce convulsions that are not always relieved by naloxone.

Despite effective analgesic benefit, extended periods of opioid treatment can result in a range of undesirable side effects, not the least of which is the development of tolerance. Tolerance and physical dependence with repeated use is a characteristic feature of most opioids. Dependence seems to be closely related to tolerance, since treatments that block tolerance to morphine also block dependence. In vivo studies in animal models demonstrate the importance of neurotransmitters and their interactions with opioid pathways in the development of tolerance to morphine. Blockade of glutamate actions by noncompetitive and competitive NMDA (N-methyl-D-aspartate) antagonists also blocks morphine tolerance (Trujillo and Akil (1991) Science 251:85; and Elliott et al. (1994) Pain 56:69). Similarly, blockade of the glycine regulatory site on NMDA receptors has similar effects to decrease tolerance (Kolesnikov, Y. et al. (1994) Life Sci. 55:1393). Administering inhibitors of nitric oxide synthase in morphine-tolerant animals reverses tolerance, despite continued opioid administration (Kolesnikov, Y. et al. (1993) Proc. Natl. Acad. Sci. U.S.A. 90:5162). These studies indicate several important aspects of tolerance and dependence. First, the selective actions of drugs on tolerance and dependence demonstrate that analgesia can be dissociated from these two unwanted actions. Second, the reversal of preexisting tolerance by NMDA antagonists and nitric oxide synthase inhibitors indicates that tolerance is a balance between activation of processes and reversal of those processes. These observations suggest that, by use of selective agonists and/or antagonists, tolerance and dependence in the clinical management of pain can be minimized or disassociated from the therapeutic effects.

Methadone is a synthetic analgesic with morphine-like properties. However, their clinical actions are distinctly different. Methadone has been demonstrated to have a lower dependence potential than morphine (Blake, A. D. et al. (1997) J. Biol. Chem. 272: 782-790), and is effectively used in the treatment of opioid addiction despite being a full agonist at the μ-receptor. Methadone has two enantiomeric forms, the D- and L-isomers. The racemic mixture (DL-methadone) is the form commonly used clinically and in laboratory studies. Both isomers have a low affinity for δ- and κ-opioid receptors (Kristensen et al., 1995 Life Sci. 56(2): PL45-50). However, an interesting caveat of both isomers of methadone is that they exhibit similar affinities for the noncompetitive binding site of the NMDA receptor in rat forebrain and spinal cord synaptic membranes. Morphine did not show affinity for this site on the NMDA receptor, indicating that this type of binding is not prototypical of all opioids. It appears that binding to the NMDA receptor does not affect the analgesic actions of methadone, as D-methadone can still bind well but fails to elicit an analgesic effect.

There have been many attempts to decrease the clinical dependence on morphine for the management of pain, not the least of which includes administering morphine with another analgesic. Conflicting reports suggest that although methadone blocks the euphoric effects of opioids, it does not interfere with the analgesic effects of morphine or meperidine (Rubenstein, R. B. et al. (1976) Am. J. Surg. 131: 566-569). Other studies have suggested that the pain management requirements can be met in methadone-maintained patients using standard doses of opioids, such as morphine or meperidine, in addition to their methadone maintenance dose. However, Fultz and Senay found that because of cross-tolerance to other opioids, most methadone-maintained patients would require more frequent administration of an analgesic than opioid naive patients, hence the total daily dose would be higher (Fultz, J. M. and Senay, E. C. (1975) Ann. Intern. Med. 82: 815-818). Similar conclusions were drawn by Doverty et al, who reported patients receiving methadone maintenance treatment were cross-tolerant to the antinociceptive effects of morphine. Further, they found that conventional doses of morphine were ineffective in managing episodes of acute pain among this patient group (Doverty, M., et al. (2001) Pain 93: 155-163). Thus, methadone and morphine appear to be acting through similar, if not the same, receptors to effect analgesia

Taber and coworkers (1969) have also studied morphine and methadone combinations. The authors concluded that morphine and methadone, when co-administered, produce only additive effects in mice that were induced by acetic acid treatment to undergo abdominal stretching (also known as a “writhing”test). Acetic acid-induced abdominal stretching assays are well known in the art to be indicative of mild pain models in humans. By using the writhing test, Taber et al. did not address the effect of methadone and morphine on moderate or severe pain, which is typically induced in mice by the radiant heat tail-flick assay. Moreover, from a historical standpoint, methadone was clinically administered as a racemic mixture, and as such, the additive effect observed by Taber et al. could indicate that the concentration of the L-isomer was not present in an amount sufficient to produce a greater effect.

While morphine remains the opioid of choice in the clinical management of pain, tolerance and dependence preclude its long-term use. Thus, there is a need in the art for a therapeutic agent that can reduce the effective dose of morphine or enhance its pharmacological effects. Many other widely used analgesics have no effect on morphine-induced analgesia or display very modest, additive effects when used in combination with morphine. Additionally, most opioid analgesics display similar effects of tolerance and dependence as morphine, with the exception of methadone. While methadone is an attractive candidate to decrease the effects of dependence and tolerance, prior evidence indicates that methadone 1) blocks the effects of morphine through cross-tolerance at the same receptor that modulates the actions of morphine, 2) displays only modest, additive effects when administered with morphine, or 3) has no effect on morphine-induced analgesia.

### SUMMARY OF THE INVENTION

Opioids are the most widely used analgesics in the treatment of severe pain, however tolerance and dependence preclude their sustained use. It has now been surprisingly shown that L-methadone, a synthetic analgesic historically used to decrease the effects of opioid addiction, exhibits synergy when administered together with another opioid analgesic.

Therefore, the present invention relates to compositions comprising therapeutic doses of L-methadone and at least one other opioid analgesic effective to produce a synergistic, antinociceptive response to moderate or severe pain.

In a preferred embodiment, the other opioid is selected from the group consisting of morphine, morphine-6-glucuronide, codeine, and 6-acetylmorphine.

In a preferred embodiment, the doses of L-methadone and an opioid effective to produce an analgesic effect are lower than the effective dose of each drug alone.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

These and other objects and embodiments are described in or are obvious from and within the scope of the invention, from the following Detailed Description.

### BRIEF DESCRIPTION OF THE DRAWINGS

FIG. 1A depicts interactions of L-methadone with other μ agonists. Dotted bars represent L-methadone alone, while open bars represent the indicated drug alone. Stacked bars represent the added effect of the two drugs given alone. The filled bars represent the co-administration of the two drugs.

FIG. 1B shows interactions of morphine with other μ agonists. Dotted bars represent morphine alone, while open bars represent the indicated drug alone. Stacked bars represent the added effect of the two drugs given alone. The filled bars represent the co-administration of the two drugs.

FIG. 2 demonstrates the effect of the D- and L-isomers of methadone on analgesia in conjunction with morphine. Dotted bars represent morphine alone, and open bars represent the D- or L-isomer of methadone together representing the expected additive effects of the two drugs. The filled bars represent the co-administration of morphine with either D- or L-methadone.

FIG. 3 depicts isobolographic analyses of combinations of L-methadone and several other μ-selective ligands. (A) Morphine is co-administered with methadone. (B) Methadone is co-administered with morphine-6-glucuronide. (C) Methadone is co-administered with codeine. (D) Methadone is co-administered with 6-acetylnorphine. (E) Methadone and fentanyl produce additive effects when co-administered.

FIG. 4 shows interactions of L-methadone/morphine with respect to analgesia and the inhibition of gastrointestinal transit.

### DETAILED DESCRIPTION OF THE INVENTION

Opioids serve an important function in providing analgesia. Synergistic potentiation of analgesia through administration of an opioid combination offers a new approach to severe and/or chronic pain management. Further, the ability to decrease tolerance using synthetic opioids with lower dependence potential, such as L-methadone, is an important advancement in the clinical treatment of pain. It has now been found that administration of threshold amounts of L-methadone together with atv least one another opioid analgesic result in the synergistic potentiation of antinociceptive responses while advantageously decreasing tolerance and dependence potential.

The present invention encompasses a pharmaceutical composition comprising L-methadone and at least one other opioid in amounts sufficient to potentiate an antinociceptive response to moderate or severe pain.

The present invention further encompasses methods of providing analgesia to a subject in need thereof comprising administering pharmaceutical composition(s) comprising L-methadone and at least one other opioid analgesic in amounts sufficient to potentiate an antinociceptive response to moderate or severe pain.

As used herein “potentiated antinociceptive response” is a pain-reducing response elicited through the synergistic effect of at least two opioids, in which the combined effect is greater than the sum of the effect produced by either opioid agent alone.

Likewise, the term “synergy” and its related terms “synergistic” and “synergism” refers to a pharmacological dose-response wherein the combined effect of two chemical agents is greater than the sum of the effect of each agent given alone. The term “additive” and its related term “additivity” refer to a combined pharmacological effect of two chemical agents that is equal to the sum of the effect of each given alone.

A “subject” refers to a vertebrate, preferably a mammal, and more preferably a human. Mammals include but are not limited to humans, farm animals, sport animals, and pets.

The term “opiate” refers to drugs derived from opium and include morphine, codeine, and a wide variety of semisynthetic congeners derived from them and from thebaine, another component of opium. The term “opioid” is more inclusive, applying to all agonists and antagonists with morphine-like activity as well as to naturally occurring and synthetic opioid peptides. The analgesics referred to herein will be referred by the more general term, “opioid”.

The present invention further comprises a pharmaceutical composition comprising L-methadone and at least one other opioid analgesic. Preferably, at least one of the other opioids in the compositions and methods of the instant invention is morphine. Other opioids are suitable, including but not limited to, compounds based on or derived from morphine-like compounds and analogs. The second opioid can be, but is not limited to, morphine, codeine, morphine-6-glucuronide, and 6-acetylmorphine. As the field of research in the present area expands, new opioids will be discovered and can be effectively used in accordance with the present invention. Preferably, the pharmaceutical composition comprises both L-methadone and at least one other opioid analgesic. Administration of each component separately is also envisioned by the present invention.

Methadone is chiral and exists in two enantiomeric forms: the D- and the L-isomer. Methadone is available and used clinically as a racemic mixture of both D- and L-isomers (DL-methadone), typically comprised of equal amounts of each isomer. Racemic mixtures having a threshold amount of L-methadone can be used in methods and compositions of the present invention. Preferably, racemic mixtures of the present invention contain L-methadone at a percentage of about 65%, 75%, 85% or 95%. Most preferably, L-methadone with insubstantial amounts of D-methadone, if any, is administered. Thus, compositions comprising pure L-methadone are highly desirable for use in methods of the present invention.

The amount of each opioid necessary to produce a therapeutic effect in an affected area is based on, or related to, the size of the area and the relative condition that is to be treated. For example, the amount of each opioid needed to treat severe pain or inflammation is likely to be greater than the amount of opioid needed to treat moderate forms of the affliction. In addition, an acute condition will likely require less medication for a shorter period of time than a chronic condition. Individual sensitivities will also influence the dosage amounts administered to a particular subject. A determination of the appropriate dose is within the skill of one in the art given the parameters herein.

In terms of the present invention, the preferred dosage ranges are determined by considering the pharmacological characteristics of each opioid, the amount of each opioid delivered, and the method of administration. L-methadone can typically be administered at a dosage of 1-60 mg, more preferably between 5-10 mg, about every 6 hours, in subjects who have not received prior administration of the analgesic (referred as “naive subjects”). L-methadone has a high oral-parenteral ratio compared to other opioids. A typical oral dose of L-methadone can be between 2-30 mg, depending on the weight of the subject. Injectible routes of administration will require a smaller dose, typically between 2-10 mg (Physicians' Desk Reference, 2001, 55^{th} Edition).

Likewise, the concentration of each therapeutic agent, including, for example the opioid in the pharmaceutical composition defined by the instant invention can be from about 1-300 mg, depending on the method of administration, the opioid administered, and the formulation of the opioid admninistered. One skilled in the art will appreciate that the dosages of codeine will typically be higher than that of morphine, while dosages of 6-acetylmorphine and morphine-6-glucuronide will generally be lower than morphine. Morphine can be administered at a dosage range between 1-60 mg, more preferably between 15 and 30 mg, every four to six hours. The variety of dosage formulations available for morphine and other opioids will be apparent to one skilled in the art and may require adjustments in dosage ranges, especially if, for example, a controlled-release formulation is used. The metabolite morphine-6-glucuronide exhibits higher potency than morphine, and can typically be administered at dosages about half of what is administered for morphine. Morphine-6-glucuronide may also be administered at dosage ranges between 1-60 mg, and preferably between 5 and 15 mg. Codeine requires higher dosages and can be administered in the range between 10-500 mg, more preferably between 100 and 300 mg. However, the active component of heroin, 6-acetylmorphine, can be administered at doses about half of that of morphine, preferably between 1-60 mg, and more preferably between 5 and 15 mg.

Preferably, the L-methadone composition is administered concomitantly with one or more opioid analgesics (e.g., in the same formulation). Where administration is sequential rather than concomitant, any order can be used. L-methadone can be administered prior to or following the opioid analgesic. Preferably, for a single sequential administration, the drugs would be administered within 1 minute to 12 hours of each other. For example, where administration is sequential, methadone can be administered every 8-12 hours with morphine every 4-6 hours.

Durations of drug action are under a wide range of control based upon the formulation. For example, immediate release morphine should be re-administered orally every 4-6 hours. However, certain fomulations require dosing every 24 hours. Since the drugs have different durations of action, preferably oral formulations that extend drug actions are used (e.g. in a fixed ratio with similar releases). For example, an immediate release composition comprising L-methadone (e.g., on the outside of the pill) with a slow release of morphine can be given every 8-12 hours. By altering the formulations, almost any combination would be possible with dosing up to every 24 hours.

The foregoing doses can be readily optimized following the teachings herein, based on known pharmacological protocol, by those of ordinary skill in the art, with no more than routine optimization. Of course, the preferred lower limit for drug delivery is that necessary to bring about an antinociceptive effect. The preferred upper limit is less than that amount which produces untoward side effects. It will be apparent to those of skill in the art that subjects who have received repeated administrations of the opioid analgesics described above will require an increase in dosage compared to naive subjects. In subjects who have developed tolerance to methadone or the other opioid analgesics described above, amounts of the analgesic can be adjusted in increments ranging from 25% to 100%.

The pharmaceutical compositions of the present inventionc can be in a variety of conventional depot forms. These include, for example, solid, semi-solid and liquid dosage forms, such as tablets, pills, powders, controlled-release preparations, liquid solutions or suspensions, liposomes, capsules, suppositories, injectible and infusible solutions. Such dosage forms may include pharmaceutically acceptable carriers and excipients well known to those skilled in the art. These carriers and excipients include, for example, RIBI, ISCOM, ion exchangers, alumina, magnesium stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes such as protamine sulfate, disodium hydrogen phosphate, sodium chloride, zinc salts, colloidal silica, magnesium trisilicate, calcium silicate, polyvinyl pyrrolidone, cellulose-based substances, glycerol, plant gums, and polyethylene glycol. Carriers and excipients for topical or gel base forms may be selected from the group consisting of sodium carboxymethylcellulose, polyacrylates, polyoxyethylene-polyoxypropylene-block polymers, polyethylene glycol, and wood wax alcohols. The preferred form of the instant invention depends upon the intended mode of administration and prophylactic application.

Any pharmaceutically acceptable mode of administration, including oral, topical, sublingual, rectal, as well as parenteral, intravenous, intramuscular, intralesional or subcutaneous injection, may be used to administer the pharmaceutical compositions defined by the present invention. For example, the composition may be administered to the subject in any pharmaceutically acceptable dosage form including those which may be administered to a patient intravenously as bolus or by continued infusion over a period of hours, days, weeks, or months, intramuscularly—including paravertebrally and periarticularly—subcutaneously, intracutaneously, intra-articularly, intrasynovially, intrathecally, intralesionally, periostally. The compositions of the invention can be in the form of a unit dose and can be administered orally or intravenously by bolus injection. However, continuous infusion can be appropriate for certain applications, afflictions, and disease states. As stated elsewhere in this specification, the preferred method of administration will depend on the prophylactic application.

Although it is not crucial, the dilution and/or formulation of the opioid combinations in a physiologically acceptable excipient or carrier can be important and useful in providing the final dosage concentration. The invention thus encompasses concentrated forms for subsequent dilution before use or sale. The compositions of the present invention can be administered by preparing a solution of the opioid combination in a solvent such as water, saline, aqueous dextrose, glycerol, ethanol or dimethyl sulfoxide (DMSO), with or without other excipients. The composition can also contain other medicinal agents, pharmaceutical agents, adjuvants, excipients, binding agents, disintegrants, fillers, coating agents, lubricants, antimicrobial agents, carriers, and auxiliary substances such as wetting or emulsifying agents, pH buffering agents and other ingredients.

The following examples are provided to illustrate, but not limit, the claimed invention.

### EXAMPLES

### Example 1

### Interactions of L-Methadone with Other μ Agonists

The relative potencies of a series of μ-opioids were determined by their effective dosage (ED₅₀) values from dose-response curves. These values are given in Table 1.

TABLE 1
ED₅₀ values of drugs alone and in combination with L-methadone

| | ED50 Value (mg/kg s.c.) | | | |
|---|---|---|---|---|
| | | Combination | | |
| | | (Total Drug Dose) | | |
| | | | Additive | Potency |
| Drug | Drug alone | Observed | (predicted) | Enhancement |
| L-Methadone | 1.9 ± 0.2 | | | |
| D-Methadone | >>4 | | | |
| Morphine | 4.7 ± 1.1 | 0.83 ± 0.12 | 3.11 ± 0.32 | 3.75 (P < 0.00 [symbol] |
| M6G | 3.7 ± 0.4 | 1.03 ± 0.34 | 2.80 ± 0.21 | 2.73 (P < 0.00 [symbol] |
| Codeine | 3.7 ± 0.4 | 0.74 ± 0.10 | 2.79 ± 0.22 | 3.78 (P < 0.00 [symbol] |
| 6-Acetylmorphine | 0.20 ± 0.03 | 0.27 ± 0.05 | 1.08 ± 0.09 | 3.94 (P < 0.00 [symbol] |
| Fentanyl | 0.021 ± 0.005 | 0.79 ± 0.09 | 0.89 ± 0.09 | 1.13 (N [symbol] |

Male CD-1 mice (25-30 g in weight) were purchased from Charles River Laboratories, Inc (Wilmington, Mass.). All drugs used were obtained from the Research Technology Branch of the National Institute on Drug Abuse (Rockville, Md.). Drugs were administered systemically via subcutaneous injections. Analgesia was assessed 30 minutes post-injection using radiant heat tail-flick assay. Baseline latencies ranged between 2.0 and 3.2 seconds. A maximal cutoff latency of 10 seconds was set to minimize tissue damage. Analgesia was assessed quantally as a doubling or greater of the baseline latency for each mouse. Quantal measurements are well known and are described in D'Amour, F. E. and Smith, D. L. (1941) J. Pharmacol. Exp. Ther. 72: 174-179; and in Pasternak, G. W. et al. (1980a) J Pharmacol. Exp. Ther. 214: 455-462. Groups of mice were compared using Fisher's exact test. ED₅₀ values and 95% confidence limits were calculated by probit analysis as described in Tallarida, R. J. (2000) Drug Synergism and Dose Effect Data Analysis. Chapman and Hall/CRC Press, Boca Raton, Fla. The statistical significance of the combinations, complete dose-response data were determined for each compound and were examined with probit regression analysis using PharmTools Pro (The McCary Group, Elkins Park, Pa.). Each compound was paired in a fixed-ratio combination with methadone to assess whether the combination displayed enhanced potency indicative of synergism.

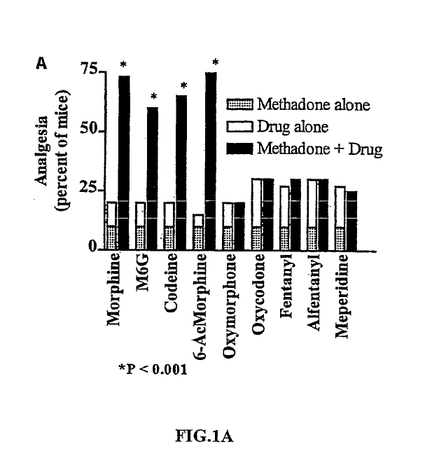

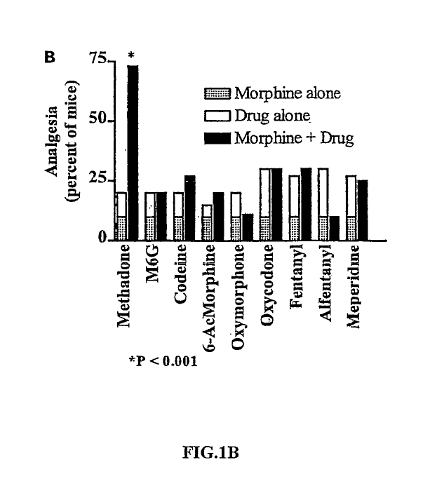

After establishing the relative potencies of these μ opioids, the interactions between a fixed dose of a number of μ opioids and a fixed dose of L-methadone were examined. Mice were tested 30 minutes after injection of L-methadone, morphine, M6G (1 mg/kg), codeine (1 mg/kg), 6-acetylmorphine (0.05 mg/kg), oxymorphone (0.01 mg/kg), oxycodone (0.5 mg/kg), or meperidine (2 mg/kg). Fentanyl (0.006 mg/kg) and alfenanyl (0.01 mg/kg) were given 20 minutes after morphine injection (10 minutes before testing) in order for the peak effects to coincide. FIG. 1A demonstrates that most of the single-dose combinations with L-methadone revealed additive interactions, including oxymorphone, oxycodone, fentanyl, meperidine, and alfentanyl. However, L-methadone gave responses with several other drugs that were far greater than anticipated. The combination of morphine and methadone clearly was greater than the sum of their independent actions (P<0.001), as was the combination of M6G with L-methadone (P<0.001). The combinations of L-methadone with either codeine (P<0.001) or 6-acetylmorphine, the active component of heroin, also generated greater than additive responses (P<0.001).

### Example 2

### Interactions of Morphine with Other μ Agonists

The same analysis as described in Example 1 was applied to administration of morphine either alone, or in combination with other μ agonists. Mice were tested 30 minutes after injection of L-methadone, morphine, M6G (1 mg/kg), codeine (1 mg/kg), 6-acetylmorphine (0.05 mg/kg), oxymorphone (0.01 mg/kg), oxycodone (0.5 mg/kg), or meperidine (2 mg/kg). Fentanyl (0.006 mg/kg) and alfenanyl (0.01 mg/kg) were given 20 minutes after morphine injection (10 minutes before testing) in order for the peak effects to coincide. Analgesia was assessed 30 minutes post-injection using radiant heat tail-flick assay. Baseline latencies ranged between 2.0 and 3.2 seconds. A maximal cutoff latency of 10 seconds was set to mnimize tissue damage. Analgesia was assessed quantally as a doubling or greater of the baseline latency for each mouse. Quantal measurements are described in D'Amour, F. E. and Smith, D. L. (1941) J. Pharmacol. Exp. Ther. 72: 174-179; and in Pastenak, G. W. et al. (1980a) J. Pharmacol. Exp. Ther. 214: 455-462.

Groups of mice were compared using Fisher's exact test. ED₅₀ values and 95% confidence limits were calculated by probit analysis as described in Tallarida, R. J. (2000) Drug Synergism and Dose Effect Data Analysis. Chapman and Hall/CRC Press, Boca Raton, Fla. The statistical significance of the combinations, complete dose-response data were determined for each compound and were examined with probit regression analysis using PharmTools Pro (The McCary Group, Elkins Park, Pa.). Each compound was paired in a fixed-ratio combination with methadone to assess whether the combination displayed enhanced potency indicative of synergism. Strikingly, morphine showed greater than additive interactions only with L-methadone. The interactions of morphine with all the other opioids tested yielded only additive effects, as described in FIG. 1B.

### Example 3

### Effect of the D- and L-Isomers of Methodone on Analgesia in Conjuction with Morphine

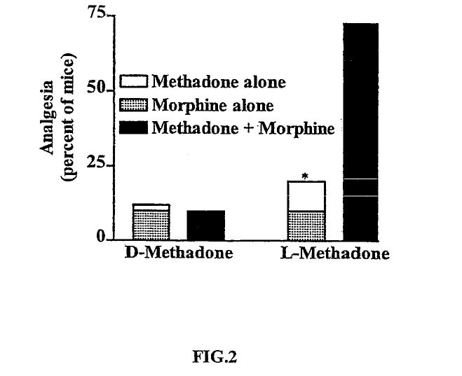

The interaction between morphine and methadone was seen only with the L-isomer of methadone. Groups of mice (n≧20) were injected subcutaneously and tested 30 minutes later for analgesia. Statistical significance was determined with Fisher's exact test. D-methadone, which has poor affinity for opioid receptors, but which does interact with NMDA receptors, did not show any effects alone at doses up to 4 mg/kg s.c., a dose corresponding to the ED₈₀ dose of L-methadone, and did not influence morphine responses when both were given together (morphine=1 mg/kg). This was surprising given that the dose of D-methadone (4 mg/kg) was 8-fold higher than the dose of L-methadone (0.5 mg/kg) that did reveal synergy. The results are depicted in graphical format in FIG. 2.

### Example 4

### Isobolographic Analysis of Combinations of L-Methodone and Several Other μ-Selective Ligands

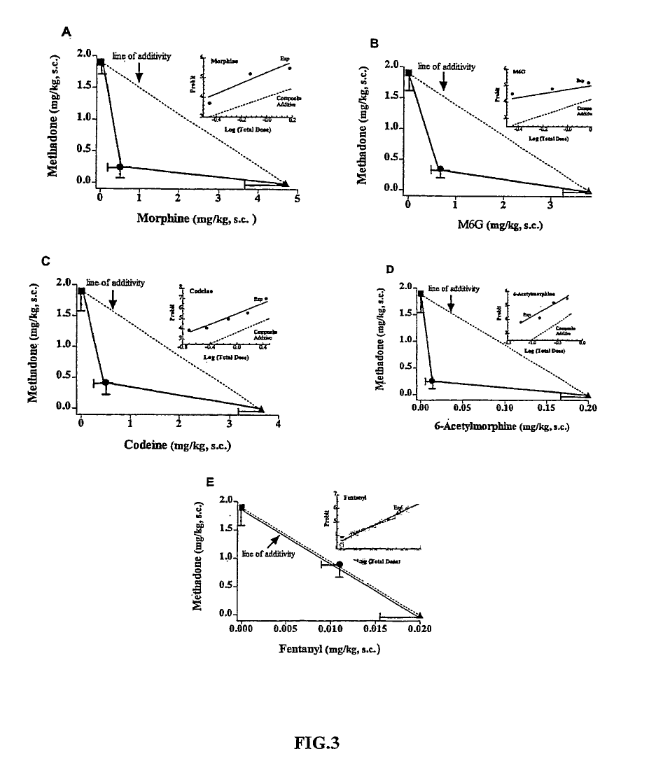

The ED₅₀ values from dose-response curves using fixed ratios of L-methadone with a series of opioids were compared to the predicted additive results to determine synergy or additivity, shown in FIG. 3. Dose-response curves were generated using fixed ratios of L-methadone with morphine (A), M6G (B), codeine (C), 6-acetylmorphine (D), or fentanyl (E). The points on the axes represent the ED50 values for either L-methadone or the second drug when each was administered alone. Combination drug points falling on or around the line connecting the ED₅₀ values alone represent additivity, whereas points falling below the line suggest synergy. In addition, statistical significance was determined by comparing the dose-response curve for the combination to the composite additive curve determined from each agent alone. The experimental dose-response, plotted as a log-probit graph, was then compared with the composite additive line using ANOVA (analysis of variance between groups). Total doses of the two drugs combined were used in the plots.

The combination of morphine and methadone was significant greater than the composite additive line (F_{2,12} =15.00; p<0.002). The combination of M6G and methadone was significantly greater than the composite additive line (F_{2,11}=17.03; p<0.001). The combination of codeine and methadone was significantly greater than the composite additive line (F_{2,14} =17.03; p<0.001). The combination of 6-acetylmorphine and methadone was also significantly greater than the composite additive line (F_{2,13}=41.6; p<0.001), while the combination of fentanyl and methadone was not significantly greater than the composite additive line (F_{2,12}=3.36).

### Example 5

### L-Methadone/Morphine Interactions on Analgesia and the Inhibition of Gastrointestinal Transit

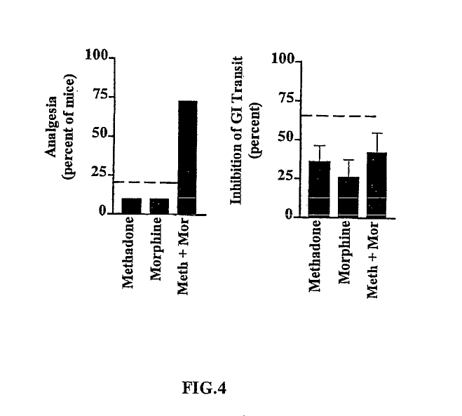

The interactions between L-methadone and morphine were examined in the context of 1) analgesia or 2) other μ opioid receptor actions, such as the inhibition of gastrointestinal transit. Both morphine and L-methadone separately inhibit gastrointestinal transit in the mouse. Groups of mice (n=10) received either L-methadone (0.25 mg/kg s.c.) or morphine (0.5 mg/kg s.c.) alone or in combination and were tested for analgesia, followed by inhibition of gastrointestinal transit 30 minutes later. The dashed lines in FIG. 4 indicate the anticipated values for additivity. The analgesic activity of the combination was significantly greater than additive (P<0.05), however inhibition of transit by the combination in the gastrointestinal transit assay was not. This observation is striking, since it suggests that using the combination of the two drugs should significantly increase the therapeutic index for analgesia compared with at least one potential side effect.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended claims.

### REFERENCES

Bare, L. A., Mansson, E., Yang, D. 1994. “Expression of two variants of the human mu opioid receptor mRNA in SK—N—SH cells and human brain” FEBS Lett. 354(2): 213-6.

Blake, A. D., Bot, G., Freeman, J. C., Reisine, T. 1997. “Differential opioid agonist regulation of the mouse mu opioid receptor” J. Biol. Chem. 272(2): 782-90.

D'Amour, F. E. and Smith, D. L. 1941. “A method for determining loss of pain sensation” J. Pharmacol. Exp. Ther. 72: 174-179.

Davis, A. M. and Inturrisi, C. E. 1999. “d-Methadone Blocks Morphine Tolerance and N-Methyl-D-Aspartate-Induced Hyperalgesia” J. Pharmacol. Exp. Ther. 289: 1048-1053.

Delfs, J. M., Kong, H., Mestek, A., Chen, Y., Yu, L., Reisine, T., Chesselet, M. F. 1994. “Expression of mu opioid receptor mRNA in rat brain: an in situ hybridization study at the single cell level” J Comp. Neurol. 345(1): 46-68.

Doverty, M., Somogyi, A. A., White, J. M., Bochner, F., Beare, C. H., Menelaou, A., Ling, W. 2001. “Methadone maintenance patients are cross-tolerant to the antinociceptive effects of morphine” Pain 93(2): 155-63.

Elliott, K., Minami, N., Kolesnikov, Y. A., Pasternak, G. W., luturrisi, C. E. 1994. “The NMDA receptor antagonists, LY274614 and MK-801, and the nitric oxide synthase inhibitor, NG-nitro-L-arginine, attenuate analgesic tolerance to the mu-opioid morphine but not to kappa opioids” Pain 56(1): 69-75.

Fultz, J. M. and Senay, E. C. 1975. “Guidelines for the management of hospitalized narcotics addicts” Ann. Intern. Med. 82(6): 815-8.

Giros, B., Pohl, M., Rochelle, J. M., Seldin, M. F. 1995. “Chromosomal localization of opioid peptide and receptor genes in the mouse” Life Sci. 56(18): PL369-75.

Kolesnikov, Y. A., Pick, C. G., Ciszewska, G., Pasternak, G. W. 1993. “Blockade of tolerance to morphine but not to kappa opioids by a nitric oxide synthase inhibitor” Proc. Natl. Acad. Sci. USA. 90(11): 5162-6.

Kolesnikov, Y. A., Maccechini, M. L., Pasternak, G. W. 1994. “1-Aminocyclopropane carboxylic acid (ACPC) prevents mu and delta opioid tolerance” Life Sci. 55(18): 1393-8.

Kristensen, K., Christensen, C. B., Christrup, L. L. 1995. “The mu1, mu2, delta, kappa opioid receptor binding profiles of methadone stereoisomers and morphine” Life Sci. 56(2): PL45-50.

Liang, Y., Mestek, A., Yu, L., Carr, L. G. 1995. “Cloning and characterization of the promoter region of the mouse mu opioid receptor gene” Brain Res. 679(1): 82-8.

Lutz, R. A. and Pfister, H. P. 1992. “Opioid receptors and their pharmacological profiles” J. Recept. Res. 12(3): 267-86.

Min, B. H., Augustin, L. B., Felsheim, R. F., Fuchs, J. A., Loh, H. H. 1994. “Genomic structure analysis of promoter sequence of a mouse mu opioid receptor gene” Proc. Natl. Acad. Sci. USA; 91(19): 9081-5.

Olson, G. A., Olson, R. D., and Kastin, A. J. 1989. “Endogenous opiates b 1988” Peptides 10(6): 1253-80.

Pasternak, G. W., Childers, S. R., and Snyder, S. H. 1980a. “Naloxazone, long-acting opiate antagonist: effects in intact animals and on opiate receptor binding in vitro” J. Pharmacol. Exp. Ther. 214: 455-462.

Pasternak, G. W. 1993. “Pharmacological mechanisms of opioid analgesics” Clin. Neuropharmacol. 16(1): 1-18.

Pasternak, G. W., Standifer, K. M. 1995. “Mapping of opioid receptors using antisense oligodeoxynucleotides: correlating their molecular biology and pharmacology” Trends Pharm. Sci. 16(10): 344-50.

Physicians'Desk Reference 2001. 55^{th} Edition, (Sifton et al. Eds) Medical Economics Company, Montvale, N.J.

Reisine, T., and Bell, G. I. 1993. “Molecular biology of opioid receptors” Trends Neurosci. 16(12): 506-10.

Reisine, T. and Pastemak G. W. 1996. Goodman & Gilman's The pharmacological basis of therapeutics, Ninth Edition (Hardman et al. eds.) McGraw-Hill pp 521-555.

Rossi, G. C., Pan, Y. X., Brown, G. P., Pasternak, G. W. 1995. “Antisense mapping the MOR-1 opioid receptor: evidence for alternative splicing and a novel morphine-6 beta-glucuronide receptor” FEBS Lett. 369(2-3): 192-6.

Rossi, G. C., Brown, G. P., Leventhal, L., Yang, K., Pasternak, G. W. 1996. “Novel receptor mechanisms for heroin and morphine-6 beta-glucuronide analgesia” Neurosci. Lett. 216(1): 1-4.

Rubenstein, R. B., Spira, I., Wolff, W. I. 1976. “Management of surgical problems in patients on methadone maintenance” Am. J. Surg. 131(5): 566-9.

Simon, E. J. 1991. “Opioid receptors and endogenous opioid peptides” Med. Res. Rev. 11(4): 357-74.

Taber, R. I., Greenhouse, D. D., Rendell, J. K., Irwin, S. 1969. “Agonist and Antagonist Interactions of Opioids on Acetic Acid-Induced Abdominal Stretching in Mice” J. Pharmacol. Exp. Ther. 169(1): 29-38.

Tallarida, R. J. 2000. Drug Synergism and Dose-Effect Data Analysis Chapman and Hall/CRC Press, Boca Raton, Fla.

Trujillo, K. A. and Akil, H. 1991. “Inhibition of morphine tolerance and dependence by the NMDA receptor antagonist MK-801” Science 251(4989): 85-7.

Wolozin, B. L., and Pasternak, G. W. 1981. “Classification of multiple morphine and enkephalin binding sites in the central nervous system” Proc. Natl. Acad. Sci. USA 78(10): 6181-5.

Zimprich, A., Simon, T., Hollt, V. 1995. “Cloning and expression of an isoform of the rat mu opioid receptor (rMOR1B) which differs in agonist induced desensitization from rMOR1” FEBS Lett 359(2-3): 142-6.

1. A pharmaceutical composition comprising L-methadone and at least one additional opioid analgesic, both effecting opioid analgesia, in amounts sufficient to potentiate a synergistic antinociceptive response.

2. The pharmaceutical composition according to claim 1, wherein the opioid analgesic is selected from the group consisting of morphine, morphine-6-glucuronide, 6-acetylmorphine, and codeine.

3. A method of providing analgesia to a subject in need thereof comprising administering a pharmaceutical composition comprising L-methadone and at least one additional opioid analgesic, wherein the pharmaceutical composition is administered in an amount and a duration sufficient to potentiate an antinociceptive response.

4. A method of providing analgesia to a subject in need thereof comprising administering a pharmaceutical composition comprising L-methadone and administering a pharmaceutical composition comprising at least one additional opioid analgesic, wherein the pharmaceutical compositions are administered in an amount and a duration sufficient to potentiate an antinociceptive response.

5. The method according to claim 4, wherein L-methadone is administered in a dosage range from about 1 mg to about 60 mg.

6. The method according to claim 4, wherein L-methadone is administered in a dosage range from about 2 mg to about 10 mg.

7. The method according to claim 4, wherein the opioid analgesic is morphine.

8. The method according to claim 7, wherein morphine is administered in a dosage range from about 0.1 mg to about 60 mg.

9. The method according to claim 7, wherein morphine is administered in a dosage range from about 1 mg to about 50 mg.

10. The method according to claim 4, wherein the opioid analgesic is morphine-6-glucuronide.

11. The method according to claim 10, wherein morphine-6-glucuronide is administered in a dosage range from about 1 mg to about 60 mg.

12. The method according to claim 10, wherein morphine-6-glucuronide is administered in a dosage range from about 5 mg to about 15 mg.

13. The method according to claim 4, wherein the opioid analgesic is codeine.

14. The method according to claim 12, wherein codeine is administered in a dosage range from about 10 mg to about 500 mg.

15. The method according to claim 14, wherein codeine is administered in a dosage range from about 100 to about 200 mg.

16. The method according to claim 4, wherein the opioid analgesic is 6-acetylmorphine.

17. The method according to claim 15, wherein 6-acetylmorphine is administered in a dosage range from 1 mg to 30 mg.

18. The method according to claim 16, wherein 6-acetylmorphine is administered in a dosage range from 7 mg to 15 mg.

19. The pharmaceutical composition according to claim 1, wherein the composition comprises a racemic mixture of DL methadone having at least 65% L-methadone.

20. The method according to claim 3, wherein the pharmaceutical composition comprises a racemic mixture of DL methadone having at least 65% L-methadone.

21. The method according to claim 4, wherein the pharmaceutical composition comprising L-methadone is a racemic mixture of DL methadone having at least 65% L-methadone.

22. The method according to claim 3, wherein the pharmaceutical composition comprises a racemic mixture of DL methadone and the antinociceptive response is therapeutic for moderate or severe pain.

23. The method according to claim 4, wherein the pharmaceutical composition comprising L-methadone is a racemic mixture of DL methadone and the antinociceptive response is therapeutic for moderate or severe pain.

24. A method for potentiating an antinociceptive response by providing analgesia from L-methadone and at least one additional opioid analgesic in a subject in need thereof, comprising administering to the subject a pharmaceutical composition comprising L-methadone and at least one additional opioid analgesic in an amount and a duration sufficient to potentiate an antinociceptive response.

25. A method for potentiating an antinociceptive response by providing analgesia from L-methadone and at least one additional opioid analgesic in a subject in need thereof, comprising administering to the subject a pharmaceutical composition comprising L-methadone and administering a pharmaceutical composition comprising at least one additional opioid analgesic, both in an amount and a duration sufficient to potentiate an antinociceptive response.
